(19) United States
(12) Patent Application Publication
(10) Pub. No.: US 2026/0053542 A1
(43) Pub. Date: Feb. 26, 2026

(54) INTRAVASCULAR LITHOTRIPSY CATHETER SYSTEM WITH CONTROLLER AND GRAPHICAL USER INTERFACE

(71) Applicants: Bolt Medical, Inc., Carlsbad, CA (US); Boston Scientific Scimed, Inc., Maple Grove, MN (US)

(72) Inventors: Christopher A. Cook, Laguna Niguel, CA (US); Eric Andrew Schultheis, San Clemente, CA (US); Rachel Lynn Troutman, Minneapolis, MN (US); Lakshmi Narayan Mishra, Carlsbad, CA (US); Paige Hartley, Carlsbad, CA (US); Christine Benson, Maple Grove, MN (US)

(73) Assignees: Bolt Medical, Inc., Carlsbad, CA (US); Boston Scientific Scimed, Inc., Maple Grove, MN (US)

(21) Appl. No.: 19/376,507

(22) Filed: Oct. 31, 2025

Related U.S. Application Data
(63) Continuation-in-part of application No. 19/068,673, filed on Mar. 3, 2025.
(60) Provisional application No. 63/561,448, filed on Mar. 5, 2024.
(60) Provisional application No. 63/880,259, filed on Sep. 11, 2025.

(51) Int. Cl. A61B 18/04; A61B 17/00; A61B 18/00; A61B 34/00

(52) U.S. Cl. CPC A61B 18/042; A61B 34/25; A61B 2017/00199; A61B 2018/0022; A61B 2018/00345; A61B 2018/00702

(57) ABSTRACT

Example catheter systems and methods for using catheter systems are disclosed. An example catheter system for treating a treatment site within or adjacent to a vessel wall or heart valve includes an energy source, a catheter including an energy guide configured to receive energy from the energy source, a system controller coupled to the energy source and a graphical user interface in communication with the system controller. Further, the graphical user interface is configured to display operational information corresponding to the operation of one or more components of the catheter system. Further, the graphical user interface is configured to communicate with the system controller to control the operation of the one or more components of the catheter system.

### RELATED APPLICATION

This application claims priority to U.S. Provisional Patent Application No. 63/880,259 filed on Sep. 11, 2025, and entitled “CATHETER SYSTEM WITH USER INTERFACE”, the entirety of which is hereby incorporated by reference for any and all purposes.

This application is a continuation-in-part of U.S. patent application Ser. No. 19/068,673 filed Mar. 3, 2025, which claims priority to U.S. Provisional Patent Application No. 63/561,448 filed on Mar. 5, 2024, and entitled “GRAPHICAL USER INTERFACE FOR INTRAVASCULAR LITHOTRIPSY CATHETER SYSTEM”, the entirety of which are hereby incorporated by reference for any and all purposes.

### TECHNICAL FIELD

The present disclosure relates to intravascular lithotripsy catheter systems and methods of using intravascular lithotripsy catheter systems.

### BACKGROUND

Vascular lesions (also referred to herein as a “treatment site”) within and/or adjacent to vessels in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Intravascular lithotripsy is a method for breaking up vascular lesions within vessels in the body. Intravascular lithotripsy utilizes a combination of pressure waves and bubble dynamics that are generated intravascularly in a fluid-filled balloon catheter. In particular, in certain implementations of an intravascular lithotripsy treatment, a high energy source is used to provide energy to an energy guide and/or an emitter in order to generate plasma and, ultimately, pressure waves as well as a rapid bubble expansion within a fluid-filled balloon to crack calcification at a treatment site within the vasculature that includes one or more vascular lesions. The associated rapid bubble formation from the plasma initiation and resulting localized fluid velocity within the balloon transfers mechanical energy through the incompressible fluid to impart a fracture force on the intravascular calcium, which is opposed to the balloon wall. The rapid change in fluid momentum upon hitting the balloon wall is known as hydraulic shock or water hammer.

Conventional intravascular lithotripsy catheter systems include user interfaces that provide minimal information to the user about the catheter system. In some instances, the user interfaces of these catheter systems only indicate the number of pulses remaining on the catheter and the balloon size of the connected device. Due to the lack of information provided by the user interface, clinicians have to rely on other external devices such as indeflator pressure gauges and stopwatches or timers to get other relevant information during an intravascular lithotripsy procedure.

### SUMMARY

An example catheter system may include an energy source configured to provide energy to an energy guide of a catheter in communication with the energy source, a system controller coupled to the energy source, and a graphical user interface in communication with the system controller, wherein the graphical user interface may be configured to display operational information corresponding to the operation of one or more components of the catheter system; and wherein the graphical user interface may be configured to communicate with the system controller to control the operation of the one or more components of the catheter system.

In addition or alternatively to any example described herein, the graphical user interface may be configured to display catheter information associated with the catheter.

In addition or alternatively to any example described herein, the catheter information may include the type of catheter.

In addition or alternatively to any example described herein, the graphical user interface may be configured to display dimensional information of a balloon of the catheter for an inflated configuration of the catheter.

In addition or alternatively to any example described herein, the graphical user interface may be configured to display emitter information corresponding to a plurality of emitters of the catheter.

In addition or alternatively to any example described herein, the emitter information may include an arrangement of the of the plurality of emitters positioned within an interior of a balloon of the catheter.

In addition or alternatively to any example described herein, the graphical user interface may be configured to display a number of shots fired from a single emitter of the catheter.

In addition or alternatively to any example described herein, the graphical user interface may be configured to display a total number of shots fired from all of a plurality of emitters of the catheter.

In addition or alternatively to any example described herein, the graphical user interface may be configured to display a number of shots remaining to be fired by each emitter of a plurality of emitters of the catheter.

In addition or alternatively to any example described herein, the graphical user interface may be configured to display the number of shots fired from a single emitter of the plurality of emitters.

In addition or alternatively to any example described herein, the graphical user interface may be configured to display a total number of shot cycles remaining for a plurality of emitters of the catheter.

In addition or alternatively to any example described herein, the graphical user interface may be configured to receive a touchscreen selection of one or more individual emitters of a plurality of emitters of the catheter.

In addition or alternatively to any example described herein, the graphical user interface may be configured to provide a visual representation of the selection of the one or more individual emitters of the plurality of emitters.

In addition or alternatively to any example described herein, the graphical user interface may be configured to display pressure information corresponding to a balloon of the catheter.

In addition or alternatively to any example described herein, the pressure information may include balloon inflation pressure.

In addition or alternatively to any example described herein, the pressure information may include balloon burst pressure.

In addition or alternatively to any example described herein, the pressure information includes a visual representation of a desired pressure range.

In addition or alternatively to any example described herein, the catheter system may include the catheter comprising a plurality of energy guides including the energy guide, a balloon, and a plurality of emitters in communication with the energy guides at a location within the balloon; and catheter fluid positioned within an interior of the balloon when the balloon is in an inflated configuration, wherein each of the plurality of emitters may be configured to generate a plasma in the catheter fluid within the interior of the balloon in response to receiving energy from the energy source.

In an example, a catheter system for treating a treatment site within or adjacent to a vessel wall or heart valve may include an energy source, a catheter including an energy guide configured to receive energy from the energy source and a balloon configured to shift between a deflated configuration and an inflated configuration, an emitter coupled to a distal end region of the energy guide and positioned within an interior of the balloon, a system controller coupled to the energy source, and a graphical user interface in communication with the system controller, wherein the graphical user interface may be configured to display emitter information corresponding to the emitter, wherein the graphical user interface may be configured to display operational information corresponding to the operation of one or more components of the catheter system, and wherein the graphical user interface may be configured to communicate with the system controller to control the operation of the one or more components of the catheter system.

In an example, a method for treating a treatment site within or adjacent to a vessel wall or heart valve may include positioning a catheter adjacent to the treatment site, the catheter comprising an energy guide and the catheter is coupled to a console including an energy source configured to provide energy to the energy guide, a system controller coupled to the energy source, and a graphical user interface in communication with the system controller, wherein the graphical user interface is configured to communicate with the system controller to control the operation of the one or more components of the console and wherein the graphical user interface is configured to permit touchscreen operation of one or more components of the catheter system; and selecting an operational component on the touchscreen.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including a system controller and a graphical user interface having features of the present invention;

FIG. 2A is a simplified illustration of one embodiment of the graphical user interface of the catheter system, the graphical user interface displaying representative data at a first time (t₁);

FIG. 2B is another simplified illustration of the graphical user interface illustrated in FIG. 2A, the graphical user interface displaying representative data at a second time (t₂) that is different than the first time (t₁);

FIG. 2C is another simplified illustration of the graphical user interface illustrated in FIG. 2A, the graphical user interface displaying representative data at a third time (t₃) that is different than each of the first time (t₁) and the second time (t₂);

FIG. 2D is another simplified illustration of the graphical user interface illustrated in FIG. 2A, the graphical user interface displaying representative data at a fourth time (t₄) that is different than each of the first, second, and third times (t₁₋₃);

FIG. 2E is a schematic illustration of an embodiment of the graphical user interface;

FIG. 2F is another simplified illustration of the graphical user interface illustrated in FIG. 2A, the graphical user interface displaying representative data at a fifth time (t₅) that is different than each of the first, second, third, and fourth times (t₁₋₄);

FIG. 2G is another simplified illustration of the graphical user interface illustrated in FIG. 2A, the graphical user interface displaying representative data at a sixth time (t₆) that is different than each of the first, second, third, fourth, and fifth times (t₁₋₅);

FIG. 2H is a schematic illustration of an embodiment of the graphical user interface of the catheter system;

FIG. 2I is a schematic illustration of an embodiment of the graphical user interface of the catheter system;

FIG. 2J is a schematic illustration of an embodiment of the graphical user interface of the catheter system;

FIG. 2K is a schematic illustration of example icons of the graphical user interface of the catheter system;

FIG. 3 is an embodiment of the graphical user interface illustrating a graph showing pressure changes over time during a therapy cycle; and

FIG. 4 is a schematic diagram of an illustrative configuration of a controller/control module and a user interface.

While embodiments of the present invention are susceptible to various modifications and alternative forms, specifics thereof have been shown by way of examples and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can occur in the heart, peripheral or central vasculature, brain, musculature, or any of the internal organs.

In various embodiments, the catheter systems and related methods disclosed herein can include a catheter configured to advance to a vascular lesion, such as a calcified vascular lesion or a fibrous vascular lesion, at a treatment site located within or adjacent to a vessel wall of a blood vessel or a heart valve within a body of a patient. As used herein, the terms “treatment site,” “intravascular lesion,” and “vascular lesion” are used interchangeably unless otherwise noted. As such, the intravascular lesions and/or the vascular lesions are sometimes referred to herein as “lesions.”

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

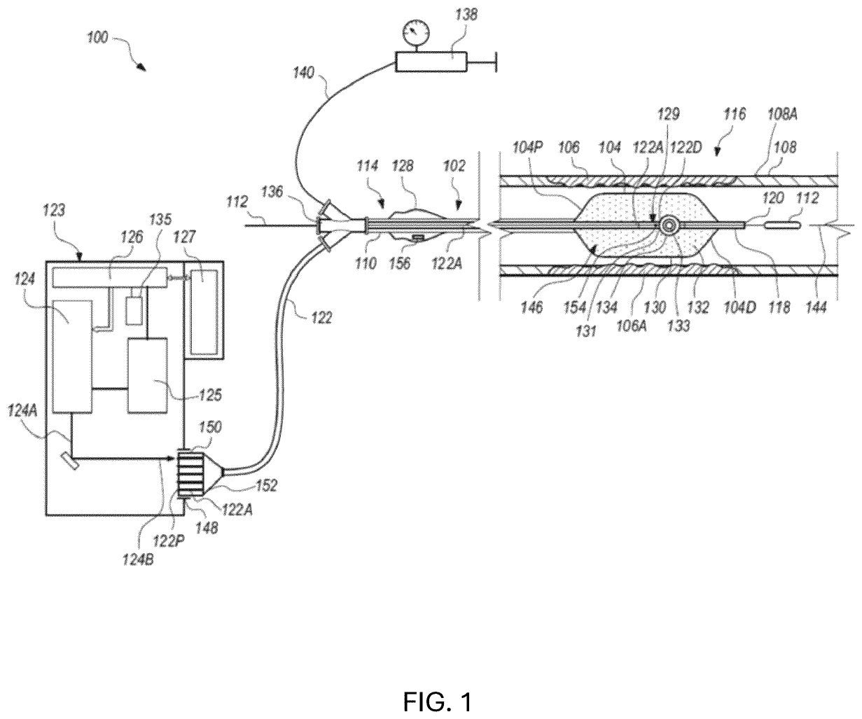

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures at one or more treatment sites within or adjacent to a vessel wall of a blood vessel or adjacent to a heart valve within a body of a patient. In the embodiment illustrated in FIG. 1, the catheter system 100 can include one or more of a catheter 102 including an energy guide bundle 122 including one or more energy guides 122A, a handle assembly 128, and an emitter assembly 129, a source manifold 136, a fluid pump 138, and a system console 123 including one or more of an energy source 124, a power source 125, a system controller 126, and a graphical user interface 127 (a “GUI”). In various embodiments, the emitter assembly 129 includes and/or incorporates at least one emitter 131 that is configured to direct and/or concentrate energy toward one or more vascular lesions 106A at a treatment site 106 within or adjacent to a vessel wall 108A of a blood vessel 108 or a heart valve within a body of a patient. Alternatively, the catheter system 100 can include more components or fewer components than those specifically illustrated and described in relation to FIG. 1.

The catheter 102 is configured to move to the treatment site 106 within or adjacent to the vessel wall 108A of the blood vessel 108 or a heart valve within the body of the patient. The treatment site 106 can include one or more vascular lesions 106A such as calcified vascular lesions, for example. Additionally, or in the alternative, the treatment site 106 can include vascular lesions 106A, such as fibrous vascular lesions. Still, alternatively, in some implementations, the catheter 102 can be used at a treatment site 106 within or adjacent to a heart valve within the body 107 of the patient 109.

The catheter 102 can include an inflatable balloon 104 (sometimes referred to herein simply as a “balloon”), a catheter shaft 110, and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118, which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118. The catheter 102 can include all of the components shown in FIG. 1 that are distal to the guide proximal end 122P, and the system console 123 can include all of the components shown in FIG. 1 that are proximal to the guide proximal end 122P.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106. It is appreciated that although FIG. 1 illustrates the balloon wall 130 of the balloon 104 being shown spaced apart from the treatment site 106 of the blood vessel 108 or a heart valve when in the inflated state, this is done for ease of illustration. It is recognized that the balloon wall 130 of the balloon 104 will typically be substantially directly adjacent to and/or abutting the treatment site 106 when the balloon 104 is in the inflated state.

As described, in various embodiments, the catheter system 100 and/or the emitter assembly 129 can include the at least one emitter 131 that is configured to transmit energy from the energy source 124 into the balloon interior 146 in order to generate plasma and/or pressure waves in the catheter fluid 132 within the balloon interior 146. Each of the emitters 131 includes a guide distal end 122D of one of the energy guides 122A, which is positioned within the balloon interior 146, and a corresponding plasma target 133 (also sometimes referred to as a “plasma generating structure” or a “plasma generator”) that is positioned near, but typically spaced apart from, the guide distal end 122D. As referred to herein, the plasma target 133 or “plasma generator” can include and/or incorporate any suitable type of structure that is located at or near the guide distal end 122D of the energy guide 122A. Energy from the energy source 124 is directed toward and received by the energy guide 122A, is guided through the energy guide 122A, and is then emitted from the guide distal end 122D of the energy guide 122A. The energy emitted from the guide distal end 122D is directed toward and impinges on and energizes the corresponding plasma target 133 for purposes of generating the plasma in the catheter fluid 132 within the balloon interior 146.

In many embodiments, the present invention utilizes a laser light source or other suitable light source as the energy source 124, and is configured to shine laser light energy onto the plasma target 133 to cause plasma generation via interaction with a plasma target material rather than optical breakdown of the catheter fluid 132. This moves the plasma creation away from the guide distal end 122D of the energy guide 122A (which can be an optical fiber in some embodiments). This can be accomplished by positioning the plasma target 133 away from the guide distal end 122D of the energy guide 122A to absorb the light energy and convert it into a plasma at some distance away from the guide distal end 122D of the energy guide 122A.

The balloon 104 suitable for use in the catheter system 100 includes those that can be passed through the vasculature of a patient when in the deflated state. In some embodiments, the balloons 104 are made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length ranging from at least three mm to 300 mm. More particularly, in some embodiments, the balloon 104 can have a length ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug-eluting coating or a drug-eluting stent structure. The drug-eluting coating or drug-eluting stent can include one or more therapeutic agents, including anti-inflammatory agents, anti-neoplastic agents, anti-angiogenic agents, and the like.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluids 132 suitable for use are biocompatible. A volume of catheter fluid 132 can be tailored by the chosen energy source 124 and the type of catheter fluid 132 used.

In certain embodiments, the catheter fluid 132 can include a wetting agent or surface-active agent (surfactant). These compounds can lower the tension between solid and liquid matter. These compounds can act as emulsifiers, dispersants, detergents, and water infiltration agents. Wetting agents or surfactants reduce surface tension of the liquid and allow it to fully wet and come into contact with optical components (such as the energy guide(s) 122A) and mechanical components (such as other portions of the emitter assembly 129). This reduces or eliminates the accumulation of bubbles and pockets or inclusions of gas within the emitter assembly 129. Nonexclusive examples of chemicals that can be used as wetting agents include, but are not limited to, Benzalkonium Chloride, Benzethonium Chloride, Cetylpyridinium Chloride, Poloxamer 188, Poloxamer 407, Polysorbate 20, Polysorbate 40, and the like. Non-exclusive examples of surfactants can include, but are not limited to, ionic and non-ionic detergents, and Sodium stearate. Another suitable surfactant is 4-(5-dodecyl) benzenesulfonate. Other examples can include docusate (dioctyl sodium sulfosuccinate), alkyl ether phosphates, and perfluorooctanesulfonate (PFOS), to name a few.

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 micrometers (μm) to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG—emission maximum=1064 nm) lasers, holmium:YAG (Ho:YAG—emission maximum=2.1 μm) lasers, or erbium:YAG (Er:YAG—emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the balloon 104. In some embodiments, each energy guide 122A can be an optical fiber, and the energy source 124 can be a laser. The energy source 124 can be in optical communication with the energy guides 122A at the proximal portion 114 of the catheter system 100.

In some embodiments, the catheter shaft 110 can be coupled to multiple energy guides 122A, such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; or six energy guides 122A can be spaced apart from one another by approximately 60 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can have a grooved outer surface, with the grooves extending in a generally longitudinal direction along the guidewire lumen 118. In such embodiments, each of the energy guides 122A and/or the emitter(s) 131 of the emitter assembly 129 can be positioned, received, and retained within an individual groove formed along and/or into the outer surface of the guidewire lumen 118. Alternatively, the guidewire lumen 118 can be formed without a grooved outer surface, and the position of the energy guides 122A and/or the emitter(s) 131 of the emitter assembly 129 relative to the guidewire lumen 118 can be maintained in another suitable manner.

The catheter system 100 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100 and/or the energy guide bundle 122 can include from one energy guide 122A to greater than 30 energy guides 122A. Alternatively, in other embodiments, the catheter system 100 and/or the energy guide bundle 122 can include greater than 30 energy guides 122A.

The energy guides 122A can have any suitable design for the purposes of generating plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter system 100 is often described with the energy source 124 as a light source, and the one or more energy guides 122A as light guides, the catheter system 100 can alternatively include any suitable energy source 124 and energy guides 122A for purposes of generating the desired plasma in the catheter fluid 132 within the balloon interior 146. For example, in one non-exclusive alternative embodiment, the energy source 124 can be configured to provide high-voltage electrical pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still, alternatively, the energy source 124 and/or the energy guides 122A can have another suitable design and/or configuration.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P to the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length of the balloon 104 and/or relative to the length of the guidewire lumen 118 to more effectively and precisely impart pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A and/or the emitter assembly 129 can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can be configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the guide distal end 122D of the energy guide 122A can include, but are not to be limited to, those having a convex surface, a gradient-index (GRIN) lens, and a mirror focus lens. Upon contact with the diverting structure, the energy is diverted within the energy guide 122A to one or more of the plasma target 133 and the photoacoustic transducer 154 that is in optical communication with a side surface of the energy guide 122A. When utilized, the plasma target 133 receives energy emitted from the guide distal end 122D of the energy guide 122A to generate plasma in the catheter fluid 132 within the balloon interior 146, which, in turn, causes the creation of plasma bubbles and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

Additionally, or in the alternative, in certain embodiments, such diverting structures that can be incorporated into the energy guides 122A, can also be incorporated into the design of the emitter assembly 129 and/or the plasma target 133 for purposes of directing and/or concentrating acoustic and mechanical energy toward specific areas of the balloon wall 130 in contact with the vascular lesions 106A at the treatment site 106 to impart pressure onto and induce fractures in such vascular lesions 106A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (also sometimes referred to generally as a “ferrule”) that houses a portion, such as the guide proximal end 122P, of each of the energy guides 122A. The guide coupling housing 150 is configured to fit and be selectively retained within the console connection aperture 148 to provide the mechanical coupling between the energy guide bundle 122 and the system console 123.

The energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 into the blood vessel 108 or the heart valve during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122, such as through the use of a multiplexer (not shown), as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma target 133 that can be located at or near the guide distal end 122D of the energy guide 122A. In particular, in such embodiments, the energy emitted at the guide distal end 122D of the energy guide 122A is directed toward and energizes the plasma target 133 to form the plasma in the catheter fluid 132 within the balloon interior 146. The plasma formation can initiate a pressure wave and can initiate the rapid formation of one or more bubbles that can rapidly expand to a maximum size and then dissipate through a cavitation event that can also launch a pressure wave upon collapse. An exemplary plasma-induced bubble 134 is illustrated in FIG. 1. The rapid expansion of the plasma-induced bubbles 134 can generate one or more pressure waves within the catheter fluid 132 and thereby impart pressure waves upon the treatment site 106. The pressure waves can transfer mechanical energy through an incompressible catheter fluid 132 to the treatment site 106 to impart a fracture force on the vascular lesions 106A at the treatment site 106. Without wishing to be bound by any particular theory, it is believed that the rapid change in catheter fluid 132 momentum upon the balloon wall 130 of the balloon 104 that is in contact with or positioned near the vascular lesions 106A at the treatment site 106 is transferred to the vascular lesions 106A to induce fractures in the vascular lesions 106A.

In various non-exclusive alternative embodiments, the sub-millisecond pulses of energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, i.e., a single pulsed source beam.

The energy sources 124 suitable for use can include various types of light sources including lasers and lamps. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths, and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kilohertz (kHz).

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to 30 MPa, or approximately at least 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, and the handle assembly 128. The power source 125 can have any suitable design for such purposes.

The system controller 126 can be electrically coupled to and receives power from the power source 125. The system controller 126 can be coupled to and is configured to control the operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate.

The system controller 126 can also be configured to control the operation of other components of the catheter system 100, such as the positioning of the catheter 102 adjacent to the treatment site 106, the inflation of the balloon 104 (including the control of pressure inside the balloon 104) with the catheter fluid 132, etc. In some embodiments, the system controller 126 can include an automatic indeflator that can be used to monitor and/or adjust pressure levels inside of the balloon 104.

Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128. The system controller 126 can query the energy source 124 to determine its operating status and conditions, control the pulse shape and energy directly, and set the pulse rate.

The system controller 126 can monitor the real-time operating parameters of the energy source 124. The operating parameters of the catheter system 100 can include energy delivery characteristics of the energy source 124 and conditions of the treatment site 106, as non-limiting, non-exclusive examples. The system controller 126 can process the output pulse characteristic data of the energy source 124. The system controller 126 can monitor pulse energy, pulse shape, and/or pulse envelope parameters of the energy source 124 using calibrated photodiode-based detectors and FPGA circuitry included in the system controller 126.

The system controller 126 can analyze data collected from the catheter 102 to adjust the operating parameters of the energy source 124 to meet target parameters. The pulse envelope shape of the energy source 124 can be controlled by the system controller 126 directly by driving current to one or more fiber amplifier stages. The energy source 124 can have a set of values for pulse energy, pulse envelope parameters (for example, pulse rise time, pulse width, top third percent, etc.), and repetition rate for each catheter 102 type, and treatment indication. The system controller 126 can improve the operation of the energy source 124 by attempting to maintain these values near their targets by dynamic adjustment upon deviation.

The system controller 126 can also monitor an energy source temperature of the energy source 124. In some embodiments, the energy source 124 can include a seed laser. Because the pulse shape of the energy source 124 can vary with the temperature of the seed laser components, monitoring and adjusting the temperature levels can lead to improved performance of the energy source 124 during treatment. In some embodiments, the system controller can use heating and cooling to adjust the seed laser operating parameters to control pulse shape.

The system controller 126 can interface directly with the energy source 124 to monitor an energy source status of the energy source 124 and control the energy source 124 through predefined commands and user-defined sets of commands. For example, in one non-limiting, non-exclusive embodiment, a command sets a drive current on a fiber preamplifier in the seed laser stage, controlling the pulse shape as it varies with energy source 124 temperature. These commands, that can be executable by the system controller 126, are automatic so that the user does not need to be involved in any processes other than treatment, simplifying the treatment process for the user.

The system controller 126 can identify when the operating parameters require adjustment. Upon identification, the system controller 126 can automatically adjust energy delivery parameters to improve lithotripsy treatment efficacy and safety based on the identified operating parameters. Based on the stage of treatment, the system controller 126 can dynamically provide a prioritized summary of the system status, treatment progress, and actionable alerts to the user based on the monitored operating parameters.

The operating parameters for the catheter 102, such as pulse energy, envelope shape, repetition rate, number of emitter stations, etc., can be stored in the system memory of the system controller 126. The system controller 126 can control the system console 123 and the energy source 124 to provide the target pulse characteristics and energize the correct energy guide 122A channels without user intervention.

The system controller 126 can be configured to automatically identify the type of catheter 102 that is used in the catheter system 100. Upon identification of the catheter type, the system controller 126 can adjust the operating parameters of the energy guides 122A and the energy source 124 based on the identified catheter type to improve performance and safety.

The system controller 126 can be configured to monitor the balloon dimensions of the inflatable balloon 104. Based on sensor feedback, the system controller 126 can adjust the balloon dimensions of the inflatable balloon in real-time to improve treatment conditions at the treatment site 106.

The system controller 126 can be configured to monitor balloon pressure of the inflatable balloon 104. For example, the system controller 126 can monitor changes in the balloon pressure of the inflatable balloon 104 as the treatment procedure progresses. The system controller 126 can automatically adjust the balloon pressure in order to improve the efficacy of the treatment procedure.

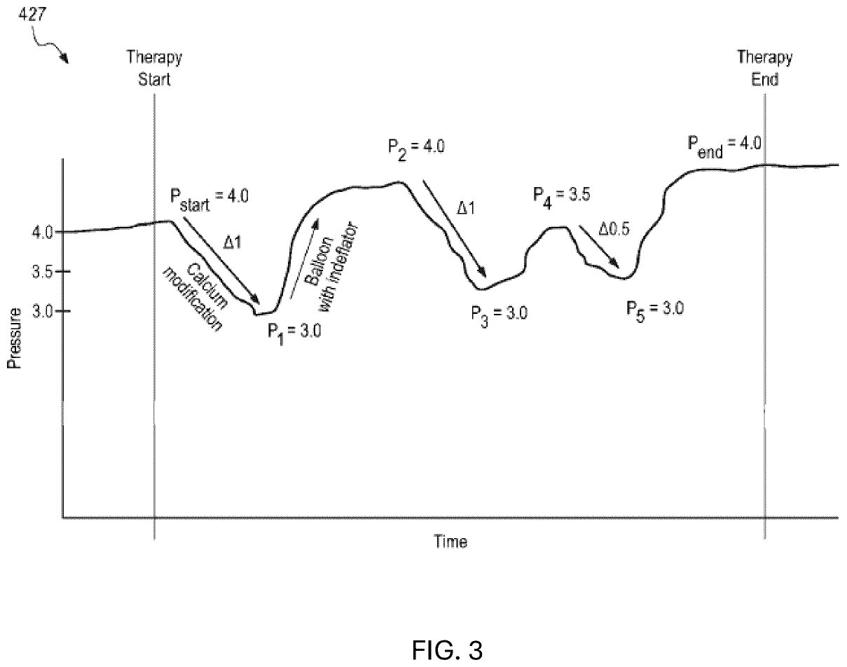

The system controller 126 can be configured to monitor and track procedure timing data during a treatment cycle, such as illustrated in FIG. 3. Among other data, the procedure timing data can include (i) an elapsed time from a beginning of a therapy cycle, and (ii) an occlusal time defined by a time period where the blood vessel 108 is occluded by the inflatable balloon 104. This information can be provided to the user via the GUI 127.

The GUI 127 is accessible by the user or operator of the catheter system 100. The GUI 127 is communicatively coupled to the system controller 126. With such a design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during, and after the use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during the use of the catheter system 100. In various embodiments, the GUI 127 can include one or more colors, different sizes, varying brightness, etc., that may act as alerts to the user or operator. Additionally, or in the alternative, the GUI 127 can provide audio data, haptic feedback, or other information to the user or operator. The GUI 127 can also provide the user or operator with control of other components of the catheter system 100. The specifics of the GUI 127 can vary depending upon the design requirements of the catheter system 100, or the specific needs, specifications, and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position, and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127. In some embodiments, the handle assembly 128 can integrate and/or include at least a portion of the system controller 126 within an interior of the handle assembly 128. For example, as shown, in certain such embodiments, the handle assembly 128 can include circuitry 156 that can form at least a portion of the system controller 126. In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

In various embodiments, as noted above, the emitter assembly 129 includes and/or incorporates the at least one emitter 131 that is configured to transmit energy from the energy source 124 into the balloon interior 146 so that plasma and/or pressure waves are generated in the catheter fluid 132 within the balloon interior. Each emitter 131 includes the guide distal end 122D of one of the energy guides 122A and the corresponding plasma target 133 that is positioned near, but typically spaced apart from, the guide distal end 122D. Additionally, in many embodiments, each emitter 131 further includes an emitter housing that is configured to maintain the desired positioning between the guide distal end 122D of the energy guide 122A and the plasma target 133, and to direct and/or concentrate energy generated in the catheter fluid 132 within the balloon interior 146 so as to impart pressure onto and induce fractures in vascular lesions 106A at the treatment site 106.

During the use of the catheter system 100, the energy guide 122A receives the energy from the energy source 124 and guides the energy from the guide proximal end 122P to the guide distal end 122D. The energy is then emitted from the guide distal end 122D of the energy guide 122A so that the energy is directed toward and impinges on and energizes the corresponding plasma target 133 for purposes of generating the plasma in the catheter fluid 132 within the balloon interior 146. The plasma generation then forms the pressure waves in the catheter fluid 132 that are directed toward the vascular lesions 106A at the treatment site 106 to provide the fracture force onto the vascular lesions 106A at the treatment site 106.

The plasma target 133 can be formed from any suitable material that is configured to generate the desired plasma in the catheter fluid 132 within the balloon interior 146 when the energy is directed from the guide distal end 122D of the energy guide 122A to impinge on the plasma target 133.

The catheter system 100 can also include a communicator 135 that is coupled to the system controller 126. The communicator 135 can be configured to provide treatment progress, treatment feedback, and system status updates to the user. The data communicated by the communicator 135 can be provided to the user via the graphical user interface 127. The design and specific features of the communicator 135 can vary to suit the design requirements of the catheter system 100.

Various alternative embodiments of GUI 127 are illustrated and described in detail below within subsequent Figures.

As with all embodiments illustrated and described herein, various structures may be omitted from the figures for clarity and ease of understanding. Additionally, the figures may include certain structures that can be omitted without deviating from the intent and scope of the invention. It is further recognized that the structures included in the various figures shown and described herein are not necessarily drawn to scale for ease of viewing and/or understanding.

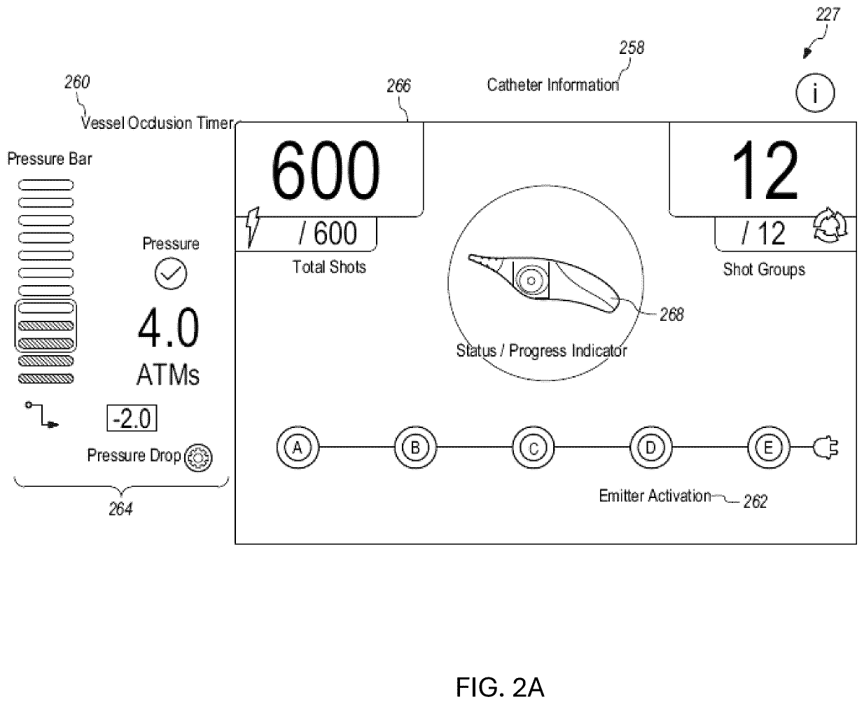

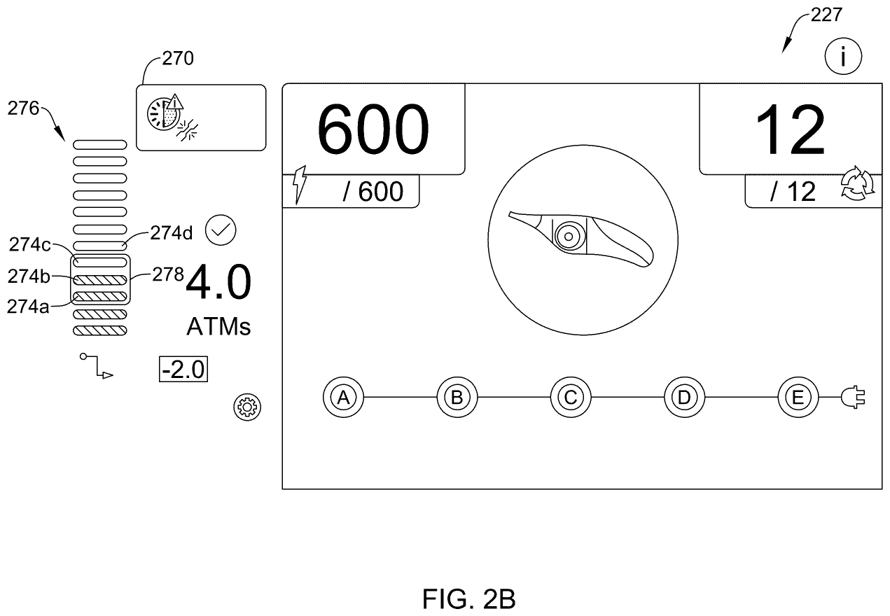

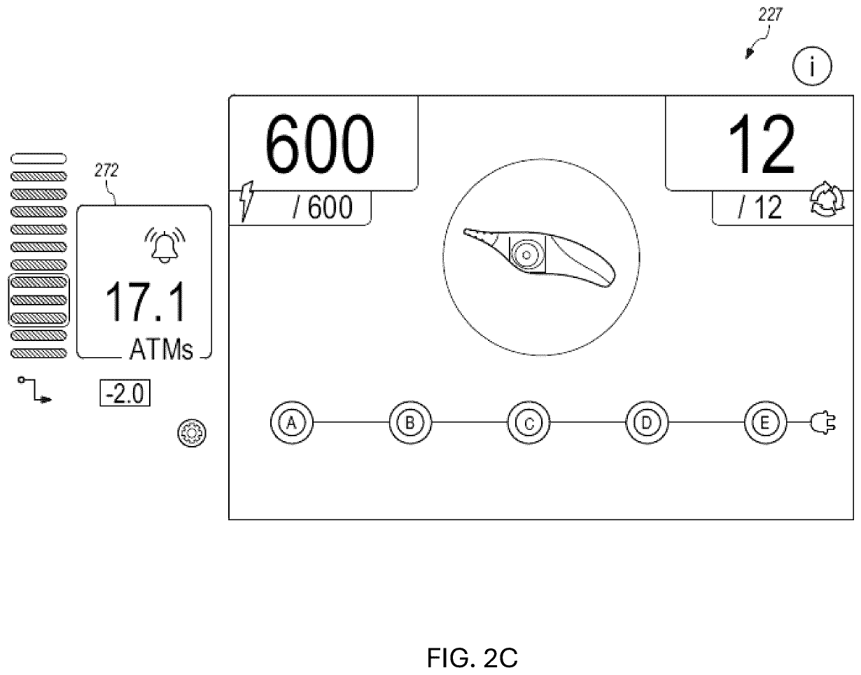

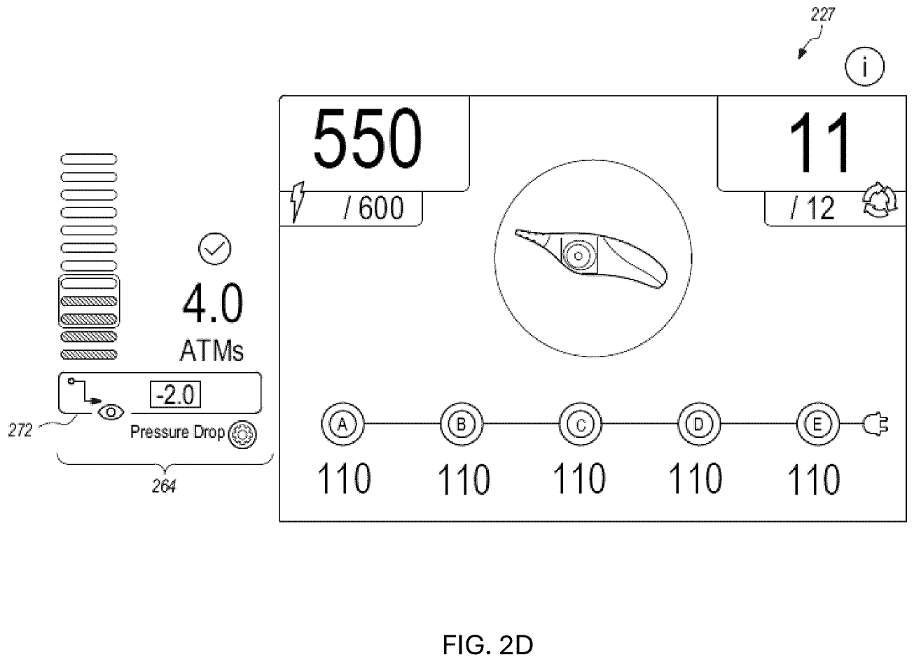

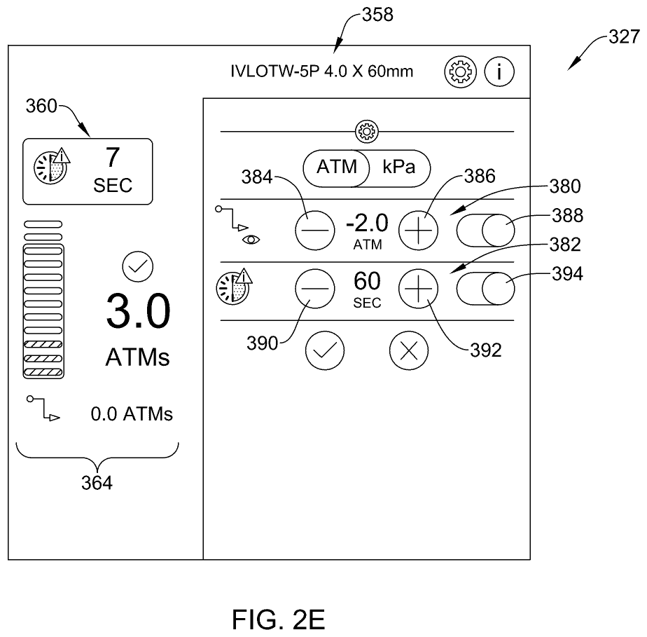

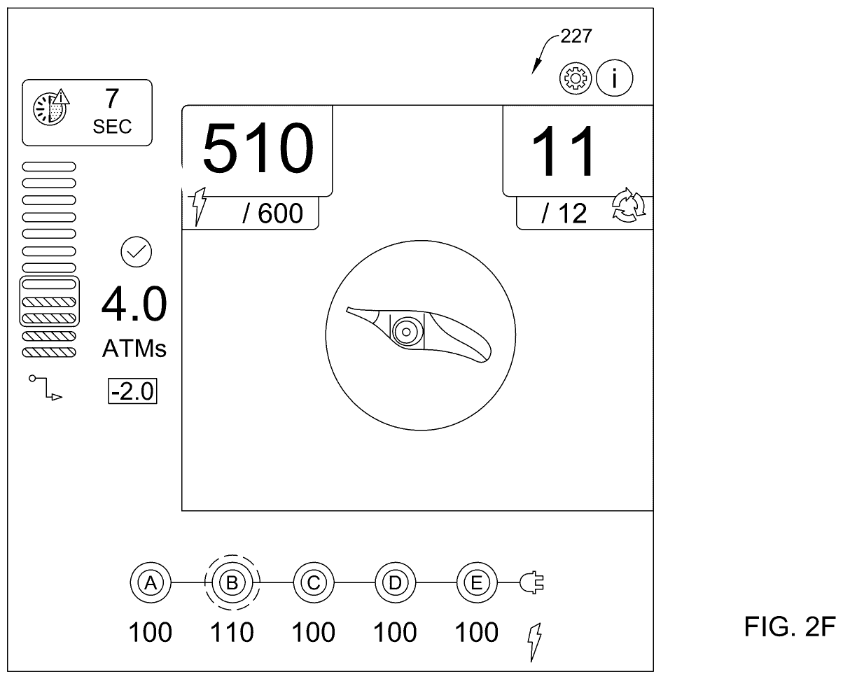

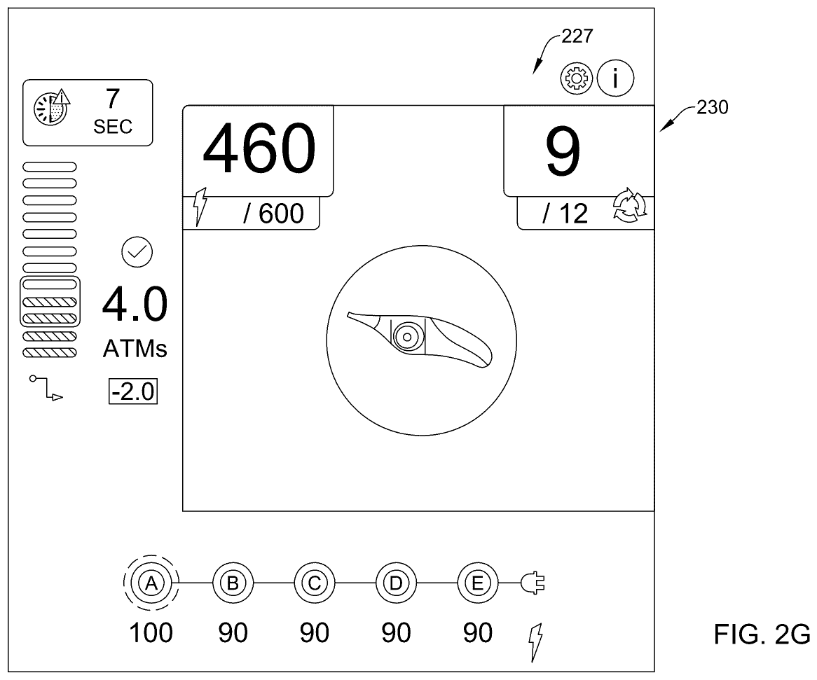

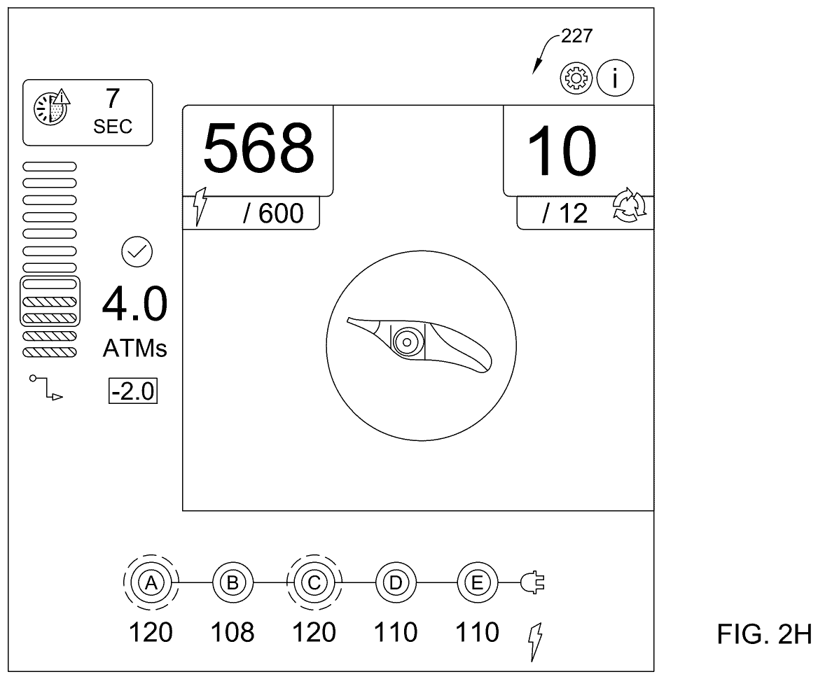

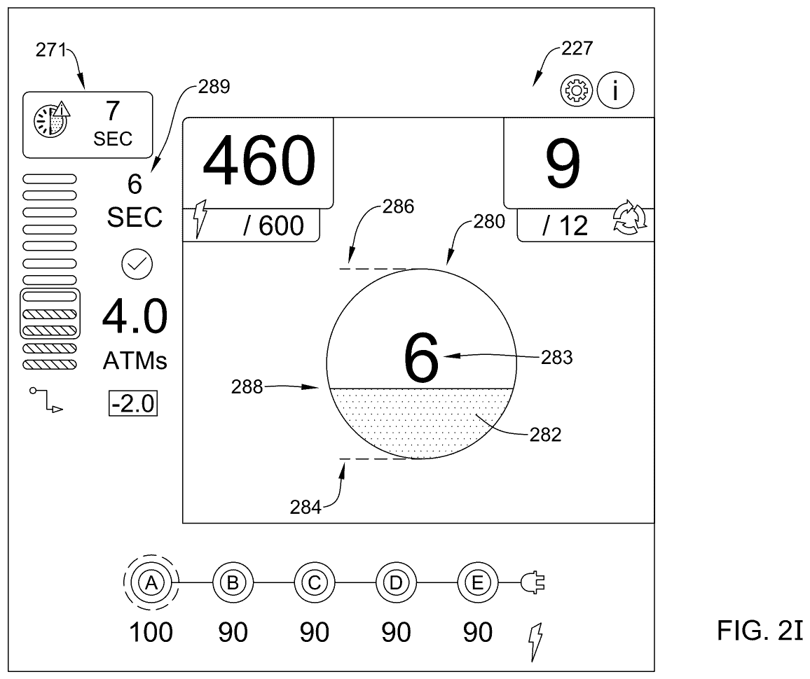

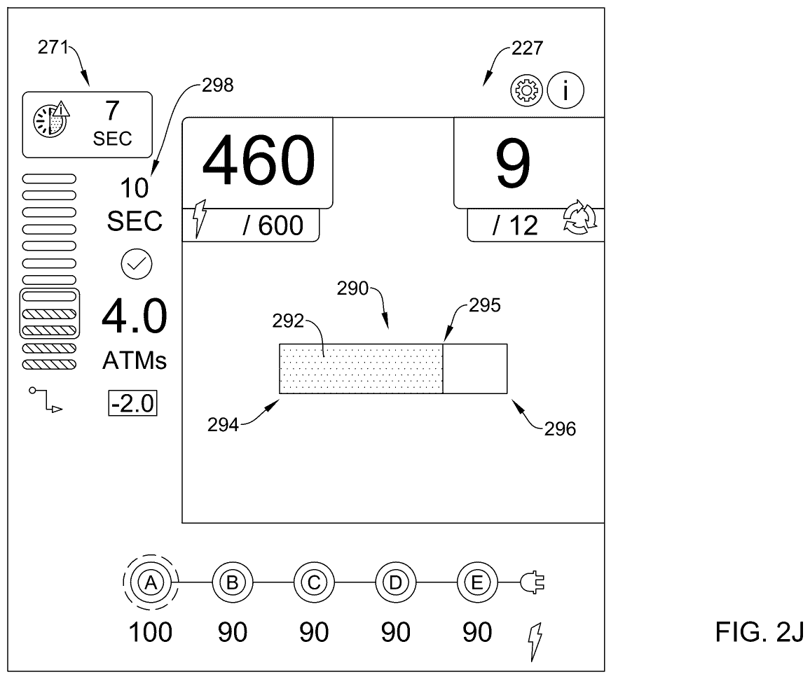

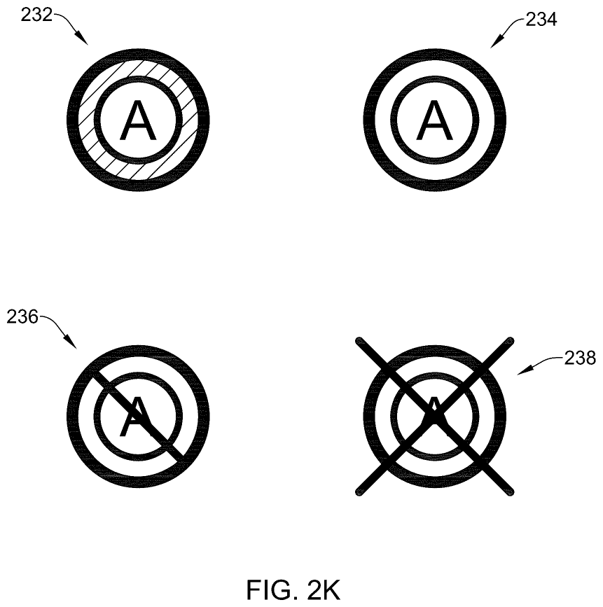

FIG. 2A is an embodiment of a graphical user interface 227 of the catheter system 100 (illustrated in FIG. 1). The graphical user interface 227 can display representative data, taken at a first time (t₁). FIG. 2A is illustrative of one possible layout of the graphical user interface 227 with the catheter 102 (illustrated in FIG. 1) inserted into the system console 123 (illustrated in FIG. 1).

In the embodiment shown in FIG. 2A, the graphical user interface 227 can include the following functional display areas: (1) catheter information 258, (2) timers 260, (3) emitter control 262, (4) pressure monitor 264, (5) shot counter 266, and (6) activation state and progress 268.

The catheter information 258 can include identification or descriptive information, etc.) for the catheter 102. For example, suitable catheter types can include above-the-knee, below-the-knee, or coronary artery disease catheters. The catheter information 258 can include emitter information such as the number of emitters 131 (illustrated in FIG. 1) and/or the individual status of each of the emitters 131. The catheter information 258 can include data, dimensions, and/or statistics of the balloon 104 (illustrated in FIG. 1). The catheter information 258 can also include cycle counting, including the number of cycles for the catheter 102 that is inserted into the system console 123 (illustrated in FIG. 1), as well as the total number of cycles remaining for the inserted catheter 102. The specifics of the catheter information 258 can vary depending upon the design requirements of the catheter system 100, or the specific needs, specifications, and/or desires of the user or operator.

The catheter information 258 can be displayed on the graphical user interface 227 so that information is displayed on the interface in a graphical or numerical format, making it easier for the user to monitor and understand the status of the catheter 102 and the progress of each treatment cycle. The user is not required to be involved with setting the important parameters that provide the target performance of a given catheter or any of the controls during operation. The user can set key operating parameters, for example, whether a given emitter station is on or off, balloon inflation, balloon pressure, and energize the catheter on or off for any given amount of time or number of cycles deemed necessary during treatment. The graphical user interface 227 can track all this information for the user in a convenient graphical or numeric format so that the user can focus on the treatment and progress/efficacy. The graphical user interface 227 can provide the catheter information 258 and controls available at a glance, thereby reducing the cognitive load on the user.

The timers 260 can include timing information such as: (i) elapsed time from the beginning of the procedure, (ii) time that the blood vessel is occluded by the balloon 104, also referred to herein as the “occluded vessel timer”, (iii) time that the balloon is maintained at a given inflation pressure, or (iv) time that an emitter fires over a cycle time period, wherein the timing information includes the elapsed time from a beginning of the cycle time period to the completion of the cycle time period. The timers 260 can also include custom timers set and/or started by the user or operator. The specifics of the timers 260 can vary depending upon the design requirements of the catheter system 100, or the specific needs, specifications, and/or desires of the user or operator.

The emitter control 262 can include individual emitter 131 selection and/or activation. The emitter control 262 can provide a visual indicator (e.g., icons, symbols, etc.) representing each individual emitter of a plurality of emitters provided by the inserted catheter 102. Referring to FIG. 2K, for example, a visual indicator representing each individual emitter may include an icon 232 which may indicate that an emitter has been activated, an icon 234 which may indicate that an emitter has been deactivated, an icon 236 which may indicate that an emitter has been disabled, and an icon 238 which may indicate that an emitter has been depleted (indicating that no pulses remain with respect to an emitter displaying the icon 238). These example icons 232, 234, 236, 238 may be utilized in place of or in addition to any of the icons shown in the example graphical user interfaces of FIGS. 2A-2D and 2F-2J disclosed herein.

The emitter control 262 can include one or more touchscreen controls for each emitter 131, allowing the user or operator to individually or collectively activate and deactivate each of the emitters 131. The system controller 126 (illustrated in FIG. 1) can be configured to identify and/or verify an emitter operational status and configuration of each of the emitters 131, including determining the number of functional emitters 131, to improve plasma generation during treatment. The specifics of the emitter control 262 can vary depending upon the design requirements of the catheter system 100, or the specific needs, specifications, and/or desires of the user or operator.

The pressure monitor 264 can include data on the pressure levels inside of the catheter 102 and/or balloon 104. The balloon 104 pressure can be displayed by the graphical user interface 227 in real-time, with indicators for desired ranges of suitable pressures for the balloon 104. The balloon 104 pressure data can be captured by a pressure sensor. In some embodiments, the pressure sensor can be located in the handle assembly 128 (illustrated in FIG. 1). The specifics of the pressure monitor 264 can vary depending upon the design requirements of the catheter system 100, or the specific needs, specifications, and/or desires of the user or operator.

The pressure monitor 264 can include a safety interlock feature that ensures the balloon 104 is inflated to a target level before the energy source 124 is activated. The pressure monitor 264 can monitor rapid pressure variations (increase or decrease) to provide indications of balloon rupturing. The pressure monitor 264 can also monitor gradual pressure changes, often in time intervals, that indicate the progress of the procedure as vessel walls become pliable, allowing the balloon 104 to expand. The pressure monitor 264 can display pressure variation over the course of a therapy cycle. The pressure monitor 264 can include a graphical display bar of the pressure, including desired pressure zones for therapy.

The shot counter 266 can include the initial number of shots for the catheter 102 that is currently inserted into the system console 123. The shot counter 266 can also include the total number of shots fired and the total number of shots remaining for the inserted catheter 102. The shot counter 266 can include an indication of the number of shots fired for each individual emitter 131 location within the catheter 102. Further, the shot counter 266 can include an indication of the number of shots remaining for each individual emitter 131 location within the catheter 102. The specifics of the shot counter 266 can vary depending upon the design requirements of the catheter system 100, or the specific needs, specifications, and/or desires of the user or operator.

The activation state and progress 268 can display the status of the catheter 102 and its availability for activation. The activation state and progress 268 can provide information on the activation of the catheter 102 as well as overall treatment progress. The specifics of the activation state and progress 268 can vary depending upon the design requirements of the catheter system 100, or the specific needs, specifications, and/or desires of the user or operator.

FIG. 2B is the graphical user interface 227 illustrated in FIG. 2A taken at a second time (t₂) that is different than the first time (t₁). The graphical user interface 227 displays representative data at the second time (t₂). FIG. 2B is illustrative of one possible layout of the graphical user interface 227 with the catheter 102 (illustrated in FIG. 1) inserted into the system console 123 (illustrated in FIG. 1).

While the first and second times (t₁₋₂) are referred to as “first” and “second” times, it is understood that these times could represent the graphical user interface 227 display data that is taken at any time that is different than another time. For example, the second time (t₂) could occur before or after the first time (t₁). It is understood that the “first,” “second,” “third,” etc. times (t_{1-X}) described herein could have any sequential order and the use of these identifiers is for purposes of identification and differentiation only and is not intended to be relevant with respect to temporal sequencing.

The embodiment illustrated in FIG. 2B includes an occluded vessel timer 270. The occluded vessel timer 270 can be activated when the inflatable balloon 104 (illustrated in FIG. 1) is inflated past a set pressure limit and obstructs or impedes blood flow in the blood vessel 108 (illustrated in FIG. 1). In other examples, the occluded vessel timer 270 may provide a visual representation and/or convey the elapsed time that the balloon 104 is inflated at a given pressure.

The occluded vessel timer 270 of the graphical user interface 227 can show the inflation pressure on the stacked bar graph 276 in digital format. For example, at the time (t₂) illustrated in FIG. 2B, the desired balloon pressure is set at 4 ATM. In certain embodiments, the readout and bar of the occluded vessel timer 270 may turn green to indicate the proximity to the desired balloon pressure. The safety interlock feature that uses catheter pressure may be disabled once the pressure is raised above a set threshold, such as 1 ATM in one non-exclusive, non-limiting embodiment. In some non-limiting, non-exclusive embodiments, the pressure readout and activation button in the handle assembly 128 (illustrated in FIG. 1) and on the graphical user interface 227 are turned from blue to green (or any suitable change in color), indicating that the catheter 102 is ready to fire or deliver therapy. The specifics of the occluded vessel timer 270 can vary depending upon the design requirements of the catheter system 100, or the specific needs, specifications, and/or desires of the user or operator.

For example, as illustrated in FIG. 2B, the stacked bar graph 276 may include bars 274a, 274b, 274c which, in some examples, may represent a desired balloon pressure range, whereby the bar 274b may represent the desired balloon pressure of 4 ATM and the bars 274a, 274c may represent balloon pressures which are lower and higher than 4 ATM, respectively. Further, in some examples, the graphical user interface 227 may include a visual indication 278 (e.g., a graphic depiction) of a desired pressure range. The visual indication 278 may take the form of a solid line which encircles a range of bars (e.g., bars 274a, 274b, 274c) representing the desired balloon pressure range.

In other examples, the stacked bar graph 276 may include one or more bars which represent the rated burst pressure of the balloon 104. For example, the bar 274d may represent the rated burst pressure of the balloon 104. Further, in some examples, visual indicator 278 may represent pressures which are approaching the rated burst pressure of the balloon 104. In such an example, the visual indicator 278 may provide a user of a visual indication (e.g., graphic depiction) that the balloon 104 is approaching, but has not yet reached, the rated burst pressure. For example, the bars 274a, 274b, 274c may sequentially light up (or change color) as the pressure increases prior to the balloon 104 reaching its rated burst pressure, which may be indicated by the lighting up or changing the color of the bar 274d.

In some examples the individual bars of the stacked bar graph 276 may represent the same or different ranges of pressures. For example, in some instances, each bar of the stacked bar graph 276 may each represent a single atmosphere of pressure. Accordingly, in some examples, the stacked bar graph 276 shown in FIG. 2B may include a pressure range of 12 atmospheres from the first (e.g., bottom bar) of the stacked bar graph 276 to the twelfth (e.g., top bar) of the stacked bar graph 276. In other examples, however, individual bars of the stacked bar graph 276 may represent different pressure ranges compared with other bars of the stacked bar graph 276. For example, one bar of the stacked bar graph 276 may represent one atmosphere, while another bar may represent a range of pressures greater than or less than one atmosphere. In some examples, the first (e.g., bottom bar) of the stacked bar graph 276 may represent one atmosphere, the eleventh bar may represent a pressure range of 11-20 atmospheres and the twelfth (e.g., top bar) of the stacked bar graph 276 may represent a pressures exceeding 20 atmospheres. This is not intended to be limiting. Rather, any of the bars of the stacked bar graph 276 may represent any pressure or range of pressures.

FIG. 2C is the graphical user interface 227 illustrated in FIG. 2A taken at a third time (t₃) that is different than each of the first time (t₁) and the second time (t₂). The graphical user interface 227 displays representative data at the third time (t₃). FIG. 2C is illustrative of one possible layout of the graphical user interface 227 with the catheter 102 (illustrated in FIG. 1) inserted into the system console 123 (illustrated in FIG. 1).

The embodiment illustrated in FIG. 2C includes an example alert 272 for pressure over a target pressure range. In some non-limiting, non-exclusive embodiments, the readout and bar graph turn yellow (or any suitable color) to indicate this condition. The specifics of the alert 272 can vary depending upon the design requirements of the catheter system 100, or the specific needs, specifications, and/or desires of the user or operator. In some examples, the alert 272 may represent that the balloon burst pressure has been reached or exceeded.

FIG. 2D is the graphical user interface 227 illustrated in FIG. 2A taken at a fourth time (t₄) that is different than each of the first, second, and third times (t₁₋₃). The graphical user interface 227 displays representative data taken at the fourth time (t₄). FIG. 2D is illustrative of one possible layout of the graphical user interface 227 with the catheter 102 (illustrated in FIG. 1) inserted into the system console 123 (illustrated in FIG. 1).

The embodiment illustrated in FIG. 2D includes one non-exclusive embodiment of an indicator for a pressure variation monitor, which is an alert 272 set by the user. For example, the pressure variation monitor can vary in color to indicate various changes in pressure. This feature can alert the user when the balloon 104 (illustrated in FIG. 1) decreases in pressure by a specified amount set by the user (at the fourth time (t₄) illustrated by FIG. 2D, a 2 atmosphere decrease was measured over a therapy cycle, one example of the therapy cycle is illustrated in FIG. 3), providing an indication that the vascular lesion 106A (illustrated in FIG. 1) in the vessel wall 108A (illustrated in FIG. 1) has been fractured allowing the blood vessel 108 to expand. This information can be displayed in the pressure monitoring 264 display location.

Alternatively, suppose the user does not want to turn the alert ‘on’. In that case, the graphical user interface 227 can display the pressure variation at the end of the therapy cycle without an alert notification. Additionally, by a user interacting with the graphical user interface 227, the system controller 126 and/or the catheter 102 can terminate the therapy cycle if a user-specified pressure variation threshold is achieved.

FIG. 2E is a schematic embodiment of a graphical user interface 327 of the catheter system 100 (illustrated in FIG. 1). FIG. 2E is illustrative of one possible layout of the graphical user interface 327 with the catheter 102 (illustrated in FIG. 1) inserted into the system console 123 (illustrated in FIG. 1).

In the embodiment shown in FIG. 2E, a graphical user interface 327 can include functional display areas: (1) catheter information 358, (2) timers 360, (3) pressure monitor 364, (4) balloon pressure variation alert adjustment selectors 380, and (5) balloon inflation time alert adjustment selectors 382.

The pressure monitor 364 may be similar in form and function to the pressure monitor 264 disclosed herein. For example, the pressure monitor 364 can include data on the pressure levels inside of the catheter 102 and/or balloon 104. The balloon 104 pressure can be displayed by the graphical user interface 327 in real-time, with indicators for desired ranges of suitable pressures for the balloon 104. The pressure monitor 364 can include a safety interlock feature that ensures the balloon 104 is inflated to a target level before the energy source 124 is activated. The pressure monitor 364 can monitor rapid pressure variation to provide indications of balloon rupturing. The pressure monitor 364 can also monitor gradual pressure changes, often in time intervals, that indicate the progress of the procedure as vessel walls become pliable, allowing the balloon 104 to expand. The pressure monitor 364 may display pressure variation over the course of a therapy cycle. The pressure monitor 364 can include a stacked bar graph 376 (similar in form and function to the stacked bar graph 276) of the balloon 104 pressure, including desired pressure zones for therapy and/or the rated burst pressure of the balloon 104.

FIG. 2E further illustrates that the graphical user interface 327 may include a timer 360. The timer 360 may be similar in form and function to the timer 270 and the timer 260 disclosed herein. The timer 360 may represent an elapsed time that the balloon 104 has been inflated to a given pressure. The timer 360 may dynamically display the elapsed time that the balloon 104 has been inflated to a given pressure. For example, the timer 360 may display the incremental counting (in seconds, for example) of the elapsed time that the balloon 104 has been inflated to a given pressure.

FIG. 2E further illustrates that the graphical user interface 327 may include desired balloon pressure variation alert adjustment selectors 380. FIG. 2E illustrates that the balloon pressure variation alert adjustment selectors 380 may include a “−” button 384 and a “+” button 386 which may permit a user to manually select a desired balloon pressure variation whereby a balloon pressure variation exceeding the selected pressure variation would trigger an alert to be displayed on the graphical user interface 327. In some examples, the balloon pressure variation alert adjustment selectors 380 may include other forms including sliders, typed inputs, text boxes, dropdown boxes/menus, selection arrows, gesture inputs, touchpad input, or the like.

Further, FIG. 2E illustrates that the balloon pressure variation alert adjustment selectors 380 may include a toggle button 388 which permits a user to choose to allow a user to manually adjust the desired balloon pressure variation alert (e.g., by toggling the toggle button 388 to an “on” position) or permitting a user to choose to allow the system controller 126 to automatically adjust the desired balloon pressure variation alert (e.g., by toggling the toggle button 388 to an “off” position).

FIG. 2E further illustrates that the graphical user interface 327 may include desired balloon inflation time alert adjustment selectors 382. FIG. 2E illustrates that the balloon inflation time adjustment alert selectors 382 may include a “−” button 390 and a “+” button 392 which may permit a user to manually select a desired balloon inflation alert time period. In some examples, the balloon inflation time adjustment alert selectors 382 may include other forms including sliders, typed inputs, text boxes, dropdown boxes/menus, selection arrows, gesture inputs, touchpad input, or the like.

Further, FIG. 2E illustrates that the balloon inflation time adjustment alert selectors 382 may include a toggle button 394 which permits a user to manually select a desired balloon inflation alert time period (e.g., by toggling the toggle button 394 to an “on” position) or permitting a user to choose to allow the system controller 126 to automatically adjust the desired balloon inflation alert time period (e.g., by toggling the toggle button 394 to an “off” position). Further, FIG. 2E illustrates the graphical user interface 327 may include a toggle button that permits a user to select the type of unit of pressure (e.g., ATM versus kPa) to be displayed on the graphical user interface 327.

FIG. 2F is the graphical user interface 227 illustrated in FIG. 2A taken at a fifth time (t₅) that is different than each of the first, second, third, and fourth times (t₁₋₄). The graphical user interface 227 displays representative data taken at the fifth time (t₅). FIG. 2F is illustrative of one possible layout of the graphical user interface 227 with the catheter 102 (illustrated in FIG. 1) inserted into the system console 123 (illustrated in FIG. 1).

The embodiment illustrated in FIG. 2F can include an emitter control 262 (shown in FIG. 2A). The emitter control 262 can be activated through a touchscreen of the graphical user interface 227 by pressing an icon (shown as emitters A-E) for the desired emitter. Using the emitter control 262, the user can select and/or deselect any number of emitters as desired to turn such emitters on/off. In this example, the second to last emitter distally, which is identified as a B, has been turned off. The deselection/off function of emitter B is illustrated in FIG. 2F as a circle shown in dashed lines, while the selection/on function of emitters A and C-D is shown with standard circles. However, as discussed herein, the form and function of the icons representing the selection and/or deselection of an emitter may be represented via a variety of different icons, including, but not limited to those disclosed in FIG. 2K.

It is appreciated that the on/off and/or selection/deselection functions of emitter selector can be illustrated/played/provided on the graphical user interface 227 using any suitable visual (e.g., the use of varying colors, shapes, shading, graphics, etc.), audio, and/or haptic feedback cues that are provided to the user. The total number of shots available for each emitter can be indicated below the emitter icons. For example, FIG. 2F illustrates the total number of shots remaining for emitters A, C, D and E is 100 shots (out of 120 initial number of shots). FIG. 2F further illustrates the total number of shots remaining for emitters B is 110 shots (out of 120 initial number of shots). Accordingly, the shot counter 266 (shown in FIG. 2A) of FIG. 2F further illustrates that 510 total shots remain out of a starting number of shots of 600, whereby 90 shots have been fired collectively across all emitters A-E. In some embodiments, the displayed number of shots available for a given emitter may decrease as the procedure progresses. Further, the displayed number of shots available for a given emitter that is turned off (e.g., deactivated) and are not fired will stay constant until that emitter is activated and fired (whereby the displayed number of shots for that respective emitter will decrease to reflect the number of shots available for that respective emitter).

FIG. 2F illustrates that, in some examples, the emitter control 262 (shown in FIG. 2A) may include icons and/or symbols which represent one or more of the emitters 131 positioned within the balloon 104. Further, the graphical user interface 227 may be configured to display the icons (shown as emitters A-E) corresponding to each of the plurality of emitters 131 as they are aligned with one another within the interior of the balloon. For example, the icon representing emitter A may be positioned distal-most within the interior of the balloon 104, the icon representing emitter E may be positioned proximal-most within the interior of the balloon 104 and the icons representing emitters B, C, D may be aligned between the emitters 131 within the interior of the balloon as arranged in FIG. 2F.

As discussed herein, using the emitter control 262, the user can select or deselect any number of emitters in any pattern as desired to turn such emitters on/off (e.g., to enable activation of a given number of emitters for firing or to deactivate emitters). For example, FIG. 2F illustrates the deactivation of emitter B (while emitters A, C, D, E remain selected and active to fire). Further, FIG. 2G illustrates another example whereby emitter A has been deactivated (while emitters B, C, D, E remain selected and active to fire). Further, FIG. 2H illustrates yet another example whereby emitters A and C have been deactivated (while emitters B, D, E remain selected and active to fire). These examples are non-limiting. Any number of emitters may be selected (e.g., activated) or deselected (e.g., deactivated) in any given arrangement or pattern.

In some examples, the visible cues (e.g., icons) representing the total number of shots available for the emitters as shown in FIGS. 2F-2J may be hidden from view until a user has interacted with the emitters (e.g., touched the touchscreen to select/deselect an emitter), whereby after the user interacts with the emitters (e.g., touches the touchscreen to select/deselect an emitter) the total number of shots available for the emitters may appear and be visible to the user. For example, FIG. 2C illustrates an example in which the total number of shots available for the emitters A-E are hidden from view. As discussed herein, after a user interacts with the emitters shown in FIG. 2C (e.g., touches the touchscreen to select/deselect an emitter A-E), the total number of shots available for the emitters may appear and be visible to the user (such as that shown in FIGS. 2F-2J).

Further, for any of the example graphical user interfaces disclosed herein, a prompt may be appear on the graphical user interface prompting the user as to whether the user would like to “confirm” a emitter selection/deselection or “cancel” an emitter selection/deselection. In some examples, the user may select/deselect any number or combination of emitters prior to choosing to “confirm” or “cancel” the selected/deselected emitters.

FIG. 2G is another example of the graphical user interface 227 illustrated in FIG. 2A taken at a sixth time (t₆) that is different than each of the first, second, third, fourth, and fifth times (t₁₋₅). The graphical user interface 227 displays representative data taken at the sixth time (t₆). FIG. 2G is illustrative of one possible layout of the graphical user interface 227 with the catheter 102 (illustrated in FIG. 1) inserted into the system console 123 (illustrated in FIG. 1) and activated.

FIG. 2G illustrates another example graphical user interface 227 which displays the number of shots available for a given emitter. Similar to that described with respect to FIG. 2F, each emitter A-E shown in FIG. 2G may have 120 total shots available to be fired (600 total shots available across all emitters A-E). As an example, FIG. 2G illustrates that emitter A has been deactivated after firing 20 shots. The other four emitters (B-E) have run through three cycles of 10 shots each (120 total shots across emitters B-E). In some examples, such as the example illustrated in FIG. 2G, the emitter A-E with the fewest shots may control the display of the cycle counter 230. In FIG. 2G, each emitter A-E has 90 shots remaining, and therefore, emitters A-E control the display of the cycle counter 230. The cycle counter may display the number of cycles (or partial cycles) remaining for the emitter with the fewest number of shots remaining to be fired. Accordingly, FIG. 2G illustrates the cycle counter 230 having been decreased by three cycles (e.g., from 12 to 9 cycles) and the total shot count decreased by one hundred forty (140) shots, as illustrated on the graphical user interface 227 shown in FIG. 2G.

In some examples, the default order of firing the emitters may be from the distal-most emitter (e.g., emitter A) to the proximal-most emitter (e.g., emitter E). Accordingly, if a user initiates the energy source to transmit pulses of energy toward the emitters, the emitters may fire in a sequence of A to B to C to D to E, whereby this cycle may repeat (e.g., A to B to C to D to E) as desired by the user. Further, if any given emitter(s) are deactivated, the order of firing of the emitters may still progress in a distal-to-proximal direction across the emitters that remain activated. For example, referring to FIG. 2H, firing of the activated emitters B, D, E may fire in a sequence of B to D to E, whereby this cycle may repeat (e.g., B to D to E) as the user continues to initiate the energy source to transmit pulses of energy toward the emitters. In some examples, if all of the emitters are deselected (e.g., no emitters are selected for activation), a message may be displayed on the graphical user interface 227 prompting a user to select an emitter.

FIG. 2H is another example of the graphical user interface 227 illustrated in FIG. 2A. The example graphical user interface shown in FIG. 2H indicates the number of shots available for emitters A-E. Similarly to that described with respect to FIG. 2F, each emitter A-E shown in FIG. 2H may have 120 total shots available to be fired (600 total shots available across all emitters A-E). As an example, FIG. 2H illustrates that emitters A and C have been deactivated and have not fired any shots. Accordingly, FIG. 2H displays the remaining shots for emitters A and C at 120. Figure H illustrates that the emitters B, D, E have fired multiple shots, whereby the firing cycle progresses from emitter B to emitter D to emitter E and back to emitter B emitter D to emitter E, etc. FIG. 2H illustrates that emitter B has fired 12 total shots, which represents having run through one complete cycles of 10 shots and a partial second cycle of 2 shots (out of 10 possible shots for the third cycle). Accordingly, FIG. 2H displays the remaining shots for emitter B is 108.

FIG. 2H further illustrates that both emitters D and E have fired 10 total shots each, which represents having completed one cycle of 10 shots each for both emitters D and E. Accordingly, FIG. 2H displays the remaining shots for emitters D and E is 110. Further, FIG. 2H illustrates the total number of shots remaining across all emitters A-E is 568 (out of 600 shots which were available at the beginning of the therapy procedure).

FIG. 2I is another example of the graphical user interface 227 illustrated in FIG. 2A. FIG. 2I illustrates that the graphical user interface 227 may include a dynamic, visual display 280 (e.g., graphical depiction) which shows the time over which a single firing cycle takes place. For simplicity, it may be assumed that the time required for each individual emitter A-E to fire a single cycle of shots (10 shots in a single cycle) may be 15 seconds. This is not intended to be limiting. Rather, the catheter system 100 may be configured for a variety of different firing cycle time periods over which emitters A-E may fire a single cycle of shots. In some examples, the length of the single firing cycle may correspond to a particular catheter 102 model being utilized with the catheter system 100.

The dynamic, visual display 280 shown in FIG. 2I may include a circle which progressively “fills up” 282 from a starting time point 284 which represents the time point at which the firing cycle starts for emitters A-E (e.g., 0 seconds in the example shown in FIG. 2I) to a cycle finish time point 286 which represents the time point at which the firing cycle is complete for emitters A-E (e.g., 15 seconds in the example shown in FIG. 2I). In other words, the dynamic, visual display 280 shown in FIG. 2I may provide a real-time visual indication of the elapsed time over which a single firing cycle takes place, whereby the empty circle of the visual display 280 “fills up” over the single firing cycle (e.g., 0 to 15 seconds in the example shown in FIG. 2I). Further, in some examples, the visual display 280 may also include a numerical representation 283 of the elapsed time of the firing cycle. For example, the numerical representation shows “6” which may indicate that a given emitter A-E has been firing for 6 seconds of a 15 second firing cycle.

Additionally, FIG. 2I illustrates that the graphical user interface 227 may include a timer 289 (distinct from the occluded vessel timer 271 shown in FIG. 2I) that provides a dynamic, real-time, numerical display of the time elapsed for a single firing cycle. The occluded vessel timer 271 may be similar in form and function to the timer 260 disclosed herein. For example, the occluded vessel timer 271 may represent an elapsed time that the balloon 104 has been inflated to a given pressure. Further, the timer 289 may correspond directly to the visual display 280, whereby the timer 289 provides the numerical time elapsed over a single firing cycle coincident with the visual display 280 “filling up” over the firing cycle. For example, the timer 289 shows “6 secs” which may indicate that emitters A-E have been firing for 6 seconds of a 15 second firing cycle. Further, the “fill line” 288 shows the amount the visual display 280 would have “filled up” over the 6 seconds of that respective firing cycle. The timer 289 and the visual display 280 will both continue to change (e.g., the time on the timer 289 increasing and visual display 280 filling up) coincident with one another as the firing cycle continues.

FIG. 2J is another example of the graphical user interface 227 illustrated in FIG. 2A. FIG. 2J illustrates that the graphical user interface 227 may include a dynamic, visual display 290 (e.g., graphical depiction) which shows the time over which a single firing cycle takes place. The visual display 290 may be similar in form and function as the visual display shown in FIG. 2I. However, FIG. 2J illustrates that instead of a circle “filling up” as shown in FIG. 2I, the visual display 290 shown in FIG. 2I may include an empty horizontal bar which “fills in” from left-to-right as viewed in FIG. 2I. The visual display 290 may include a blank or empty rectangle which progressively “fills in” 292 from a starting time point 294 which represents the time point at which the firing cycle starts (e.g., 0 seconds in the example shown in FIG. 2J) to a cycle finish time point 296 which represents the time point at which the firing cycle is complete (e.g., 15 seconds in the example shown in FIG. 2J). In other words, the dynamic, visual display 290 shown in FIG. 2J may provide a real-time visual indication of the elapsed time over which a single firing cycle takes place, whereby the empty rectangle of the visual display 290 “fills in” over the single firing cycle (e.g., 0 to 15 seconds in the example shown in FIG. 2J).

Additionally, FIG. 2J illustrates that the graphical user interface 227 may include a timer 298 (distinct from the occluded vessel timer 271 shown in FIG. 2J) that provides a dynamic, real-time, numerical display of the time elapsed for a single firing cycle taking place. The occluded vessel timer 271 may be similar in form and function to the timer 260 disclosed herein. For example, the occluded vessel timer 271 may represent an elapsed time that the balloon 104 has been inflated to a given pressure. Further, the timer 298 may correspond directly to the visual display 290, whereby the timer 298 provides the numerical time elapsed over a single firing cycle coincident with the visual display 290 “filling in” over the firing cycle. For example, the timer 298 shows “10 secs” which may indicate that emitters A-E have been firing for 10 seconds of a 15 second firing cycle. Further, the “fill line” 295 shows the amount the visual display 290 would have “filled in” over the 10 seconds of that respective firing cycle. The timer 298 and the visual display 290 will both continue to change (e.g., the time on the timer 298 increasing and visual display 290 filling in) coincident with one another as the firing cycle continues.

FIG. 3 is another embodiment of the graphical user interface 427, illustrating a graph showing pressure changes over time during a therapy cycle. As used herein, the “therapy cycle” is understood to mean the time while the treatment activation is active. In other words, as shown in FIG. 3, the therapy cycle is the time from the “Therapy Start” to the “Therapy End.”

Therapy Start can indicate when the treatment activation starts. For example, the treatment activation can start when a user of the catheter system 100 (illustrated in FIG. 1) engages a button on a handle assembly 128 (illustrated in FIG. 1).

Therapy End can indicate when the treatment activation ends. For example, the treatment activation can end when a user of the catheter system 100 releases the button on the handle assembly 128. In other embodiments, the treatment activation can end when the maximum number of energy pulses is reached.

The pressure monitor 264 (shown in FIGS. 2A-2F) of the graphical user interface 227 (shown in FIG. 2A-2F) can illustrate the change in pressure (e.g., as shown in FIG. 3, P₁/P₂/P₃/P₄/P₅−Pₛₜₐᵣₜ) at any point during the therapy cycle. At the Therapy End, the pressure monitor 264 can illustrate the change in pressure from Therapy Start to Therapy End (e.g., as shown in FIG. 3, P_{end}−Pₛₜₐᵣₜ) as a summary until the next treatment activation. In the example shown in FIG. 2F, this summary is shown as −2.0 ATM. In contrast, the therapy cycle shown in FIG. 3 would result in no change in pressure, e.g., 0 ATM.

In certain embodiments, the pressure monitor 264 can accumulate all of the pressure changes across the therapy cycle. For example, assuming the pressure change from Pₛₜₐᵣₜ to P₁ is −1 ATM but is −2.5 ATM at P_{end}. In other words, a 1 ATM decrease from the start to P₁, a decrease of 1 ATM from P₂ to P₃, and a final decrease of 0.5 ATM from P₃ to P₄. By displaying these pressure changes in each stage (e.g., P₂ to P₃), a decrease in pressure in the inflatable balloon 104 (illustrated in FIG. 1) during a treatment activation indicates that calcium is being modified. Conversely, an increase in pressure in the inflatable balloon 104 indicates the user is applying more pressure to the balloon 104 to compensate for the variation in pressure. All of this information can be displayed in the pressure monitor 264 on the graphical user interface 127.

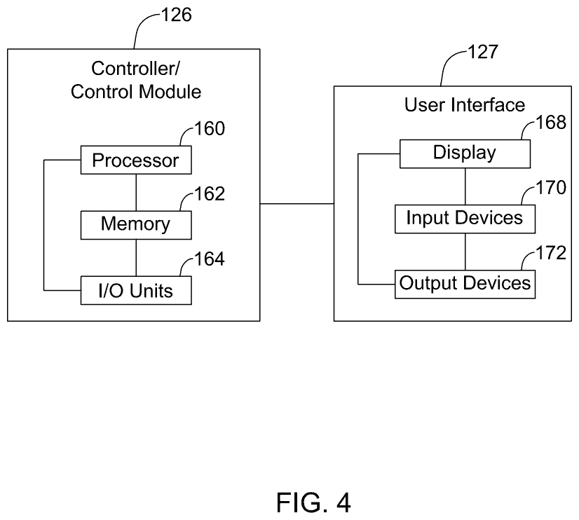

FIG. 4 depicts a schematic diagram of an illustrative configuration of the controller/control module 126 (e.g., a computing device) and a user interface 127 of the system 100. The controller 126 may be and/or may include any suitable computing device configured to process data of or for the system 100 and, in some examples, a power connector and/or electrical conductors. In some cases, one or more components of the system 100 may be incorporated into the controller 126 and/or the user interface 127. Further, one or more components of the system 100 may incorporate one or more computing devices similar to or having components similar to the controller 126 and/or the user interface 127. The controller 126 may be in communication with a wall power source, a battery power source, a renewable energy power source, and/or other suitable source of power.

The controller 126 may communicate with a remote server or other suitable computing device. When the controller 126, or at least a part of the controller 126, is a component separate from a structure of the console 123 and/or catheter 102, the controller 126 may communicate with electronic components of the system 100 over one or more wired or wireless connections or networks (e.g., LANs and/or WANs).

The controller 126 may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs) or System On Chips (SOCs), software, hardware, firmware, or any combination of these and/or other components. Although the controller 126 may be referred to herein in the singular, the controller 126 may be implemented in multiple instances, distributed across multiple computing devices, instantiated within multiple virtual machines, and/or the like.

The illustrative controller 126 may include, among other suitable components, one or more processors 160, memory 162, and/or one or more I/O units 164. Example other suitable components of the controller 126 that are not specifically depicted in FIG. 4 may include, but are not limited to, communication components, a touch screen, selectable buttons, a housing, and/or other suitable components of a controller. As discussed above, one or more components of the controller 126 may be separate from the components of the system 100 and/or incorporated into the components of the system 100.

The processor 160 of the controller 126 may include a single processor or more than one processor working individually or with one another. The processor 160 may be configured to receive and execute instructions, including instructions that may be loaded into the memory 162 and/or other suitable memory. Example components of the processor 160 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 162 of the controller 126 may include a single memory component or more than one memory component each working individually or with one another. Example types of memory 162 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 162 may be or may include a transitory or non-transitory computer readable medium. The memory 162 may include instructions stored in a transitory and/or non-transitory state on a computer readable medium that may be executable by the processor 160 to cause the processor 160 to perform one or more of the methods and/or techniques described herein.

The I/O units 164 of the controller 126 may include a single I/O component or more than one I/O component each working individually or with one another. Example I/O units 164 may be or may include any suitable types of mechanical communication hardware, electrical communication hardware, optical communication hardware, and/or software including, but not limited to, power input ports to receive power from a power source, power output ports to provide power to the console 123 and/or catheter 102, device ports for coupling with the console 123 and/or catheter 102, communication ports configured to communicate with electronic components of the system 100 and/or with other suitable computing devices or systems. Example types of I/O units 164 may include, but are not limited to, wired power components, wired optical components, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless power components, wireless optical components, wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 164.

The user interface 127 may be configured to communicate with the controller 126 via one or more wired or wireless connections. The user interface 127 may include one or more display devices 168, one or more input devices 170, one or more output devices 172, and/or one or more other suitable features.

The display device 168 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 170 may be and/or may include any suitable components and/or features for receiving user input via the user interface. Example input device(s) 170 include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s) 172 may be and/or may include any suitable components and/or features for providing information and/or data to users and/or other computing components. Example output device(s) 172 include, but are not limited to, displays, speakers, vibration systems, tactile feedback systems, optical outputs, cables, lights, and/or other suitable output devices.

The present technology is also directed toward methods for treating a treatment site within or adjacent to a vessel wall, with such methods utilizing the devices disclosed herein.

In summary, based on the various embodiments of the present invention illustrated and described in detail herein, the catheter systems and related methods can include a catheter configured to advance to a vascular lesion, such as a calcified vascular lesion, or a fibrous vascular lesion, at a treatment site located within or adjacent a blood vessel within a body of a patient. The catheter includes a catheter shaft, and an inflatable balloon that is coupled and/or secured to the catheter shaft. The balloon can include a balloon wall that defines a balloon interior. The balloon can be configured to receive a catheter fluid within the balloon interior to expand from a deflated state suitable for advancing the catheter through a patient's vasculature, to an inflated state suitable for anchoring the catheter in position relative to the treatment site.

In certain embodiments, the catheter systems and related methods utilize an energy source, e.g., a light source such as a laser source or another suitable energy source, which provides energy that is guided by one or more energy guides, e.g., light guides such as optical fibers, which are disposed along the catheter shaft and within the balloon interior of the balloon to create a localized plasma in the catheter fluid that is retained within the balloon interior of the balloon. The energy guide can be used in conjunction with a plasma generator that is positioned at or near a guide distal end of the energy guide within the balloon interior of the balloon located at the treatment site. The creation of the localized plasma can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles (also sometimes referred to simply as “plasma bubbles”) can generate one or more pressure waves in the catheter fluid retained within the balloon interior of the balloon and thereby impart pressure waves onto and induce fractures in the vascular lesions at the treatment site within or adjacent to the blood vessel wall within the body of the patient. In some embodiments, the energy source can be configured to provide sub-millisecond pulses of energy, e.g., light energy, to initiate the plasma formation in the catheter fluid within the balloon to cause the rapid bubble formation and to impart the pressure waves upon the balloon wall at the treatment site. Thus, the pressure waves can transfer mechanical energy through an incompressible catheter fluid to the treatment site to impart a fracture force on the intravascular lesion. Without wishing to be bound by any particular theory, it is believed that the rapid change in catheter fluid momentum upon the balloon wall that is in contact with the intravascular lesion is transferred to the intravascular lesion to induce fractures to the lesion.

It should be noted that, as used in this specification and the appended claims, the singular forms “a,” “an,” and “the” include plural referents unless the content and/or context clearly dictates otherwise. It should also be noted that the term “or” is generally employed in its sense, including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may be issued from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions, and sub-combinations thereof. It is, therefore, intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A catheter system, comprising:
an energy source configured to provide energy to an energy guide of a catheter in communication with the energy source;
a system controller coupled to the energy source; and
a graphical user interface in communication with the system controller;
wherein the graphical user interface is configured to display operational information corresponding to the operation of one or more components of the catheter system;
wherein the graphical user interface is configured to communicate with the system controller to control the operation of the one or more components of the catheter system.

2. The catheter system of claim 1, wherein the graphical user interface is configured to display catheter information associated with the catheter.

3. The catheter system of claim 2, wherein the catheter information includes the type of catheter.

4. The catheter system of claim 1, wherein the graphical user interface is configured to display dimensional information of a balloon of the catheter for an inflated configuration of the catheter.

5. The catheter system of claim 1, wherein the graphical user interface is configured to display emitter information corresponding to a plurality of emitters of the catheter.

6. The catheter system of claim 5, wherein the emitter information includes an arrangement of the of the plurality of emitters positioned within an interior of a balloon of the catheter.

7. The catheter system of claim 1, wherein the graphical user interface is configured to display a number of shots fired from a single emitter of the catheter.

8. The catheter system of claim 1, wherein the graphical user interface is configured to display a total number of shots fired from all of a plurality of emitters of the catheter.

9. The catheter system of claim 8, wherein the graphical user interface is configured to display a number of shots remaining to be fired by each emitter of a plurality of emitters of the catheter.

10. The catheter system of claim 9, wherein the graphical user interface is configured to display the number of shots fired from a single emitter of the plurality of emitters.

11. The catheter system of claim 1, wherein the graphical user interface is configured to display a total number of shot cycles remaining for a plurality of emitters of the catheter.

12. The catheter system of claim 1, wherein the graphical user interface is configured to receive a touchscreen selection of one or more individual emitters of a plurality of emitters of the catheter.

13. The catheter system of claim 12, wherein the graphical user interface is configured to provide a visual representation of the selection of the one or more individual emitters of the plurality of emitters.

14. The catheter system of claim 1, wherein the graphical user interface is configured to display pressure information corresponding to a balloon of the catheter.

15. The catheter system of claim 14, wherein the pressure information includes balloon inflation pressure.

16. The catheter system of claim 14, wherein the pressure information includes balloon burst pressure.

17. The catheter system of claim 14, wherein the pressure information includes a visual representation of a desired pressure range.

18. The catheter system of claim 1, further comprising:
the catheter comprising a plurality of energy guides including the energy guide, a balloon, and a plurality of emitters in communication with the energy guides at a location within the balloon; and
catheter fluid positioned within an interior of the balloon when the balloon is in an inflated configuration,
wherein each of the plurality of emitters are configured to generate a plasma in the catheter fluid within the interior of the balloon in response to receiving energy from the energy source.

19. A catheter system for treating a treatment site within or adjacent to a vessel wall or heart valve, the catheter system comprising:
an energy source;
a catheter including an energy guide configured to receive energy from the energy source and a balloon configured to shift between a deflated configuration and an inflated configuration;
an emitter coupled to a distal end region of the energy guide and positioned within an interior of the balloon;
a system controller coupled to the energy source;
and
a graphical user interface in communication with the system controller;
wherein the graphical user interface is configured to display emitter information corresponding to the emitter;
wherein the graphical user interface is configured to display operational information corresponding to the operation of one or more components of the catheter system;
wherein the graphical user interface is configured to communicate with the system controller to control the operation of the one or more components of the catheter system.

20. A method for treating a treatment site within or adjacent to a vessel wall or heart valve, the method comprising:
positioning a catheter adjacent to the treatment site, the catheter comprising an energy guide and the catheter is coupled to a console including:
an energy source configured to provide energy to the energy guide;
a system controller coupled to the energy source; and
a graphical user interface in communication with the system controller;
wherein the graphical user interface is configured to communicate with the system controller to control the operation of the one or more components of the console; and
wherein the graphical user interface is configured to permit touchscreen operation of one or more components of the catheter system; and
selecting an operational component on the touchscreen.
